(19) United States
(12) Patent Application Publication
(10) Pub. No.: US 2024/0342319 A1
(43) Pub. Date: Oct. 17, 2024

(54) DIAGNOSTIC AND THERAPEUTIC RADIOPHARMACEUTICALS FROM TRIMERIC AMINO ACID MATRIX

(71) Applicant: The Curators of the University of Missouri, Columbia, MO (US)

(72) Inventors: Kattesh V. Katti, Columbia, MO (US); Penelope Bouziotis, Athens (GR)

(21) Appl. No.: 18/624,659

(22) Filed: Apr. 2, 2024

Related U.S. Application Data
(60) Provisional application No. 63/496,028, filed on Apr. 14, 2023.

(51) Int. Cl. A61K 51/08; A61B 6/03

(52) U.S. Cl. CPC A61K 51/08; A61B 6/037

(57) ABSTRACT

A diagnostic and therapeutic radiopharmaceutical includes or consisting of a trimeric amino acid structured peptide with a radiolabel. The radiolabel can be [99mTc] [Tc(H2O)3(CO)3]⁺ or alpha emitting 225Ac. The trimeric amino acid structured peptide can be formed around phosphorus. The trimeric amino acid can be one of a Glycine, Alanine and Arginine amino acids.

### PRIORITY CLAIM AND REFERENCE TO RELATED APPLICATION

The application claims priority under 35 U.S.C. § 119 and all applicable statutes and treaties from prior U.S. provisional application Ser. No. 63/496,028, which was filed Apr. 14, 2023.

### TECHNICAL FIELD

A field of the invention concerns diagnostic and therapeutic radiopharmaceuticals. Example applications of the invention include biomedical applications such as disease treatment and disease detection.

### BACKGROUND

Most of the small molecule drugs used for diagnostic and therapeutic applications (for the diagnosis and therapy) of various diseases are based upon simple two dimensional (2D) or monolayer architectures. The mismatch of two-dimensional drug molecules with the three dimensional microenvironments of intricate cell-cell and cell-tissue matrix interactions can result in inadequate and inefficient drug-cell or drug-tissue/drug DNA interactions. Such mismatch of three-dimensional biological vectors (cells, tissue and DNA) with simple two-dimensional drug geometries has created incomplete representations of interactions of drugs to cells or tissue interactions within the complex biological environment, thus making it difficult to accurately predict in vivo drug efficacy and toxicity.

It is well-known that a variety of amino acids serve as important nutrients for the growth and proliferation of cancer cells. In order to meet the increased demand for biomass accumulation and to sustain redox homeostasis, tumor cells manifest diversion of glucose metabolism through upregulation of amino acid metabolism. Mitochondrial dysfunction of cancer cells results in loss of the ability to synthesize essential, as well as specific non-essential, amino acids adequately to support their rapid growth, metastases and proliferation. This results in increased demand from cancer cells for amino acids through upregulation of amino acid transporters which are over expressed on the surface of tumor cells. For example, two amino acid transporters, SLC7A5 and SLC7A11, have been shown to be essential for the growth and proliferation of breast tumor cells [1].

Katti et al, U.S. Pat. No. 5,948,386, describes a method to form phosphine-amine conjugates includes reacting a hydroxymethyl phosphine group of an amine-free first molecule with at least one free amine group of a second molecule to covalently bond the first molecule with the second molecule through an aminomethyl phosphorus linkage. See, also Katti et al., “Characterization of supramolecular (H₂O)₁₈ water morphology and water-methanol (H₂O)₁₅(CH₃OH)₃ clusters in a novel phosphorus functionalized trimeric amino acid host,” J Am Chem Soc. 25 (23): 6955-61 (2003).

US Published Patent Application No. US20120134918 describes trimeric alanine conjugated to Gum Arabic coated ¹⁹⁸Au nanoparticles for cancer therapy. The trimeric alanine is attached to the Gum Arabic coated 198 Au nanoparticles, which reduces availability of the trimeric alanine for bonding to metal or to form in vivo stable conjugates.

### SUMMARY OF THE INVENTION

A preferred embodiment provides a diagnostic and therapeutic radiopharmaceutical that includes or consists of a trimeric amino acid structured peptide with a radiolabel. The radiolabel can be [99mTc] [Tc(H2O)3 (CO)3]⁺ or alpha emitting 225Ac. The trimeric amino acid structured peptide can be formed around phosphorus. The trimeric amino acid can be one of a Glycine, Alanine and Arginine amino acids.

### BRIEF DESCRIPTION OF THE DRAWINGS

FIG. 1 shows a preferred trimeric amino acid that is used in present diagnostic and therapeutic radiopharmaceuticals;

FIGS. 2A-2C (Prior Art) are examples of trimeric peptides respectively based on Alanine, Arginine and Glycine, amino acids;

FIGS. 3A-3B respectively show a multidentate L-Trimeric Alanine peptide and D-Trimeric Alanine peptide;

FIG. 3C shows the structural representation of preferred Trimeric D- and L-Alanine Peptide diagnostic and therapeutic radiopharmaceutical including a [^{99m}Tc] [Tc(H₂O)₃ (CO)₃]⁺ radiolabel;

FIG. 4 is data of Accumulation of a preferred Tc-Carbonyl radiolabeled Trimeric D-Alanine peptide diagnostic and therapeutic radiopharmaceutical in various organs and tissues of breast tumor bearing mice at each time point; it also depicts Tumor-to-Blood and Tumor-to-Muscle ratios at each time point;

FIG. 5 is data of Accumulation of Tc-Carbonyl radiolabeled Trimeric L-Alanine peptide diagnostic and therapeutic radiopharmaceutical in various organs and tissues of breast tumor bearing mice at each time point; it also depicts Tumor-to-Blood and Tumor-to-Muscle ratios at each time point;

FIGS. 6A-6B are respective SPECT images of breast tumors showing clear accumulation of (FIG. 6A) Tc-Carbonyl conjugate of Trimeric D-Alanine Peptide diagnostic and therapeutic radiopharmaceutical and (FIG. 6B) Tc-Carbonyl conjugate of Trimeric: L-Alanine Peptide diagnostic and therapeutic radiopharmaceutical;

FIGS. 6C-D are tables of the percentage of the injected dose of the present diagnostic and therapeutic radiopharmaceutical which accumulates in each organ, after intravenous injection via the tail vein of each mouse, as estimated after in vivo imaging experiments.

### DETAILED DESCRIPTION OF THE PREFERRED EMBODIMENTS

A preferred embodiment is a diagnostic and therapeutic radiopharmaceutical including or consisting of a trimeric amino acid structured peptide with a radiolabel. A preferred radiolabel is a tumor-specific technetium-99m, a particular preferred radiolabel is [^{99m}Tc] [Tc(H₂O)₃ (CO)₃]⁺. Another preferred radiolabel is alpha emitting ²²⁵Ac. The trimeric amino acid is preferably one of a Glycine, Alanine and Arginine amino acid. Preferred radiopharmaceuticals provide a powerful method for Single-Photon Emission Computerized Tomography (SPECT) imaging.

In a preferred embodiment, the trimeric alanine amino acid conjugates with Technetium carbonyl to produce Technetium labeled Trimeric alanine amino acid. Techentium-99m is a diagnostic isotope that emits gamma rays for cancer imaging applications. No nanoparticle formation occurs, which opens the role of trimeric alanine amino acid to serve as a chelate (compound that binds to a metal) to produce in vivo-stable conjugates for medical applications. The diagnostic and therapeutic radiopharmaceutical can serve as a powerful cancer specific diagnostic agent because alanine targets alanine receptors found in abundance in breast tumors.

A preferred trimeric amino acid is consistent with:

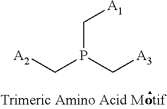

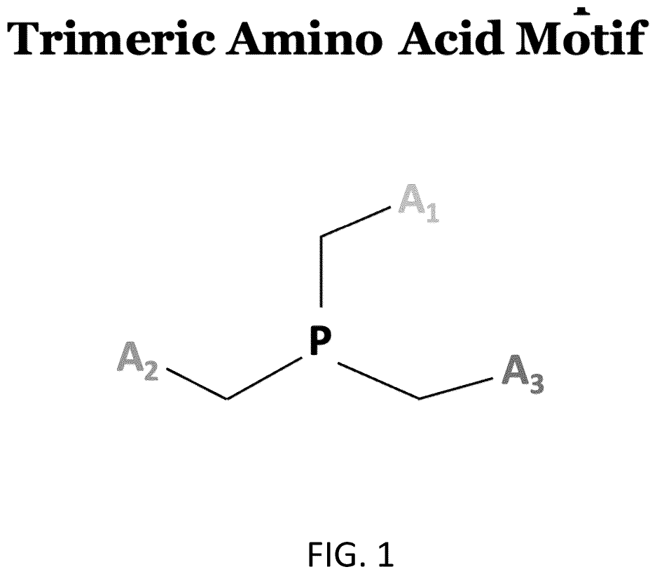

Preferred diagnostic and therapeutic radiopharmaceuticals include three-dimensional or trimeric amino acid subunits, in particular a trimeric amino acid matrix as shown in FIG. 1 or substantial equivalents, where A₁=A₂=A₃ for glycine variants, the alanine variants, and the arginine variants. Other variants can includes amino acids formed, for example, via methods disclosed in U.S. Pat. No. 5,948,386. In FIG. 1, P is Phosphorus, and each A represents an amino acid. In the examples of experiments discussed below A₁=A₂=A₃. Three versions are discussed in the experiments below, a trimeric alanine, (3 alanines bound to a central phosphorus) the trimeric arginine (3 arginines bound to a central phosphorus) and the trimeric glycine (3 glycines bound to a central phosphorus). To the knowledge of the inventors, such a trimeric amino acid structures have never been utilized in SPECT imaging as radiotherapy agents. Preferred radiopharmaceuticals include tumor-specific technetium-99m or Ac-225 alpha emitters.

A preferred method provides optimum accumulation of technetium-99m within tumors for applications as diagnostic agents through SPECT imaging of various tumors. Preferred radiopharmaceuticals are three dimensional multidentate peptides that include Ac-225 alpha emitters and provide alpha emitting cancer therapeutic agents.

Preferred embodiments of the invention will now be discussed with respect to experiments and drawings. Broader aspects of the invention will be understood by artisans in view of the general knowledge in the art and the description of the experiments that follows.

The Active Form of Tumor Necrosis Factor (TNF) is a trimer. For example, it is known that the human TNF dissociates into monomers upon addition of the nonionic detergent Triton X-100. Isolated monomers from the trimeric TNF exhibit low binding affinity (KO=70 nM) and reduced cytotoxicity. In sharp contrast, trimeric TNF exhibited high binding affinity (KO=90 PM) and cytotoxicity.

FIG. 1 shows a preferred three-dimensional Peptide trimeric amino acid motif that is part of a preferred diagnostic and therapeutic radiopharmaceutical.

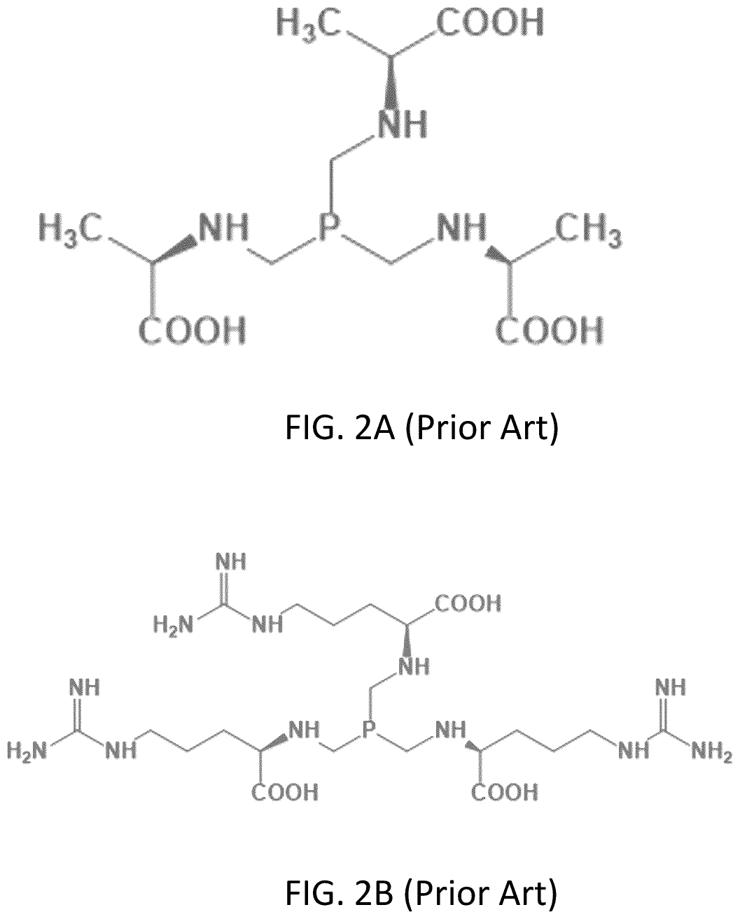

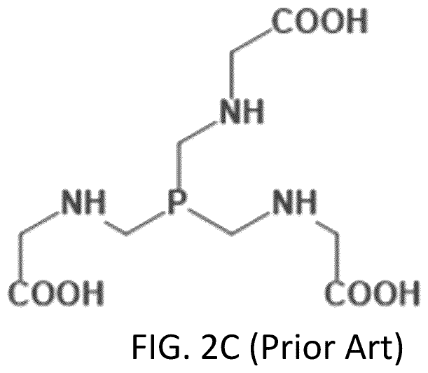

FIGS. 2A-2C are examples of trimeric peptides respectively based on Alanine, Arginine and Glycine, amino acids.

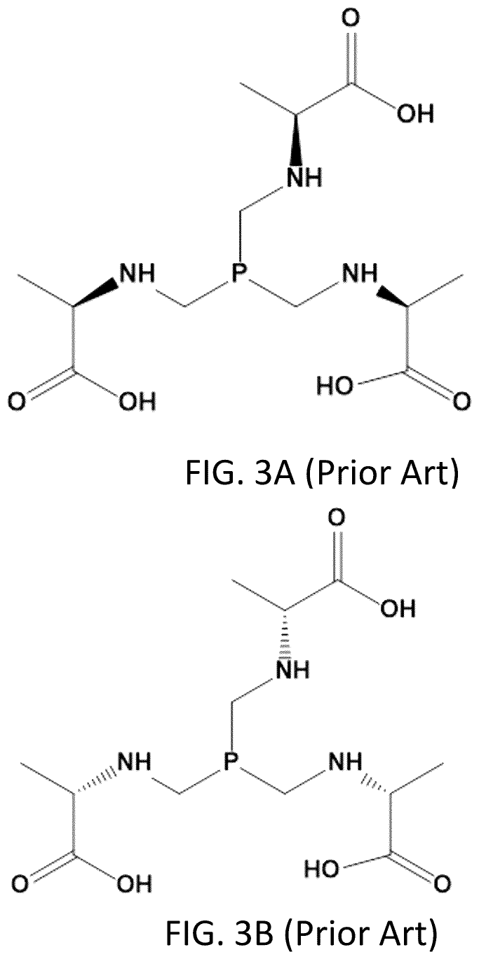

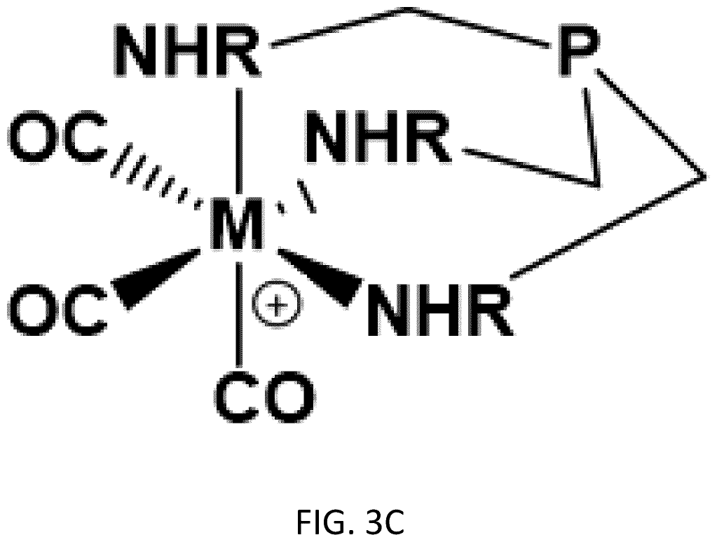

FIGS. 3A-3B respectively show a multidentate L-Trimeric Alanine peptide and D-Trimeric Alanine peptide.

Trimeric amino acid structures as shown in FIGS. 2A-2C are not believed to have been previously utilized in the constitution of trimeric Peptides or Proteins. Trimeric amino acids architectures disposed in three dimensions (X, Y and Z axis, as shown in FIG. 1) are an advantageous departure from traditional two-dimensional drug molecules.

FIG. 3C shows the structural representation of preferred Trimeric D- and L-Alanine Peptide diagnostic and therapeutic radiopharmaceutical including a [^{99m}Tc] [Tc(H₂O)₃ (CO)₃]⁺ radiolabel.

The present inventors have determined that the exogenous amino supply-dependent tumor biological characteristics of tumor cells presents unprecedented opportunities for leveraging the present diagnostic and therapeutic radiopharmaceutical. A preferred radiolabeling strategy of amino acids is based on a trimeric amino acid structural motif because trimeric amino acid, as shown in FIGS. 2A-2C and 3A-3B, was determined by the inventors to efficiently promote in vivo stable conjugations with both diagnostic and therapeutic radio probes. These peptides further promote receptor-mediated intracellular delivery of radiolabeled drugs into tumor cells. We provide experiments demonstrating trimeric D- and L Alanine (FIGS. 3A-3B), conjugated in experiments, for radiolabeling with a SPECT imaging Tc-99m-Tricarbonyl probe. Radiolabeling protocols and preclinical tumor targeting of ⁹⁹m Tc(CO)₃-labeled with both trimeric D- and L Alanine ‘Peptides’ are discussed.

### Experiments

Two radiolabeling methods were evaluated for D- and L-Trimeric Alanine, the direct radiolabeling method where Tc(VII) is reduced to Tc(V) in the presence of tin chloride SnCl₂ and radiolabeling via the tricarbonyl precursor [^{99m}Tc] [Tc(H₂O)₃ (CO)₃]⁺. Radiolabeling yield was >92% for both radiolabeling methods for both D- and L-Trimeric Alanine. However, serum stability studies showed that radiolabeling via [^{99m}Tc] [Tc(H₂O)₃ (CO)₃]⁺ afforded a more stable product in the presence of serum. Therefore, animal studies were performed for [^{99m}Tc] [Tc(H₂O)₃ (CO)₃]⁺ labeled D- and L-Trimeric Alanine. Both the D and L radiolabeled peptides exhibited fast in vivo kinetics and rapid renal excretion. Higher tumor: blood and tumor: muscle ratios at 60 min p.i. were observed for [^{99m}Tc] Tc-D-trimeric Alanine peptide, in comparison to [^{99m}Tc] Tc-L-trimeric Alanine peptide.

### Methods

Both D- and L-Trimeric Alanine were prepared according to published procedures [1,2]. After preparation, both D- and L-Trimeric Alanine were radiolabeled with Technetium-99m (^{99m}Tc), either after the reduction of Na^{99m}TcO+ with SnCl₂ or via the tricarbonyl precursor [^{99m}Tc] [Tc(H₂O)₃ (CO)₃]⁺. The prepared diagnostic and therapeutic radiopharmaceutical were then evaluated for their radiolabeling efficiency and stability in the presence of PBS and human serum. Finally, biodistribution studies were performed in tumor-bearing NOD-SCID mice bearing 4T1 breast cancer xenografts, to compare the in vivo kinetics of the radiolabeled amino acids.

Radiolabeling of D-Alanine and L-Alanine Peptide-[^{99m}Tc] [Tc(H₂O)₃ (CO)₃]⁺ diagnostic and therapeutic radiopharmaceuticals.

D-Alanine and L-Alanine (55 μL, concentration 1 mg/mL) were radiolabeled with 100 μL [^{99m}Tc] [Tc(H₂O)₃ (CO)₃]⁺ (added activity 0.8-2 mCi) by incubation at 75° C. for 1 h. The percentage of radiolabeling yield was determined by radio-TLC using ITLC-SG as the stationary phase and acetone as the mobile phase. Five μL of the labeled complex were placed at the application point of an ITLC-SG strip (1.5×10 cm), which was then allowed to dry. The free (uncomplexed) [^{99m}Tc] [Tc(H₂O)₃ (CO)₃]⁺ migrates to the front (R_{f}=0.8-1.0) of the ITLC strip leaving the labeled complex ([^{99m}Tc] Tc-D/L Alanine at the origin (Rf=0.0−0.2). Radiolabeling yield and bench stability results can be seen in Table 1 (below).

| | Radiolabeling | Bench stability |
|---|---|---|
| Sample | yield | (24 h) |
| D-Ala | 95.62 ± 2.08 | 92.34 ± 1.81 |
| L-Ala | 95.32 ± 3.23 | 94.48 ± 2.56 |

Serum stability of radiolabeled D- and L-Alanine diagnostic and therapeutic radiopharmaceutical was assessed up to 24 h post-incubation and was found to be >92% for both radiolabeled complexes.

### Biodistribution in 4T1 Tumor-Bearing Mice.

Animals used for the biodistribution studies were obtained from the breeding facilities of the Institute of Biosciences and Applications, NCSR “Demokritos”. Our experimental animal facility is registered according to the Greek Presidential Decree 56/2013 (Reg. Number: EL 25 BIO 022), in accordance with the European Directive 2010/63 which is harmonized with national legislation, on the protection of animals used for scientific purposes. All applicable national guidelines for the care and use of animals were followed. The study protocol was approved by the Department of Agriculture and Veterinary Service of the Prefecture of Athens (Protocol Number: 1607/11-04-2018). These studies have been further approved by our Institutional ethics committee and the procedures followed are in accordance with institutional guidelines.

### Cell Cultures.

4T1 cells (murine mammary carcinoma) were grown in RPMI-1640 Medium (RPMI) of pH 7.0 to 7.4, supplemented with fetal bovine serum (FBS) to a final concentration of 10%, 100 U/mL of penicillin, 2 mM glutamine, and 100 μg/mL of streptomycin. Cell cultures were maintained in flasks and were grown at 37 C in a humidified atmosphere of 5% CO2 in air. Subconfluent cells were detached using a 0.25% trypsin-0.53 mM ethylenediaminetetraacetic acid (EDTA) solution, and the subcultivation ratio was 1:8-1:10.

### Animal Models

For the animal experiments, athymic NOD-SCID mice were used. The animals were housed in air-conditioned rooms under a 12 h light/dark cycle and allowed free access to food and water. For the development of experimental tumors, 1×10⁶ 4T1 mouse mammary carcinoma cells were subcutaneously inoculated into the front left limb of SCID mice. Studies were carried out on the 6th day after cell inoculation.

### Ex Vivo Biodistribution Studies

The biological behavior D-Alanine and L-Alanine diagnostic and therapeutic radiopharmaceutical having the [^{99m}Tc] [Tc(H₂O)₃ (CO)₃]⁺ label was evaluated in SCID mice (15-25 g) bearing 4T1 tumors (n=3-5 animals per time-point). According to the experimental protocol, 100 μl/˜100 μCi of radiolabeled peptides were counted in a dose calibrator and administered to the animals via the tail vein. At 5, 15, 30 and 60 min post injection, the animals were euthanized and the organs and tissues of interest (including the tumor) were collected, weighed and measured in an automatic gamma counter. For the calculation of the injected dose in each animal, the radioactivity remaining in the tail was subtracted and a standard solution was used. All measurements were corrected for background and radioactive decay. Finally, the accumulation of the radiolabeled peptides in organs and tissues at each time point was expressed as the mean percentage of Injected Dose per gram±standard deviation (% ID/g±SD).

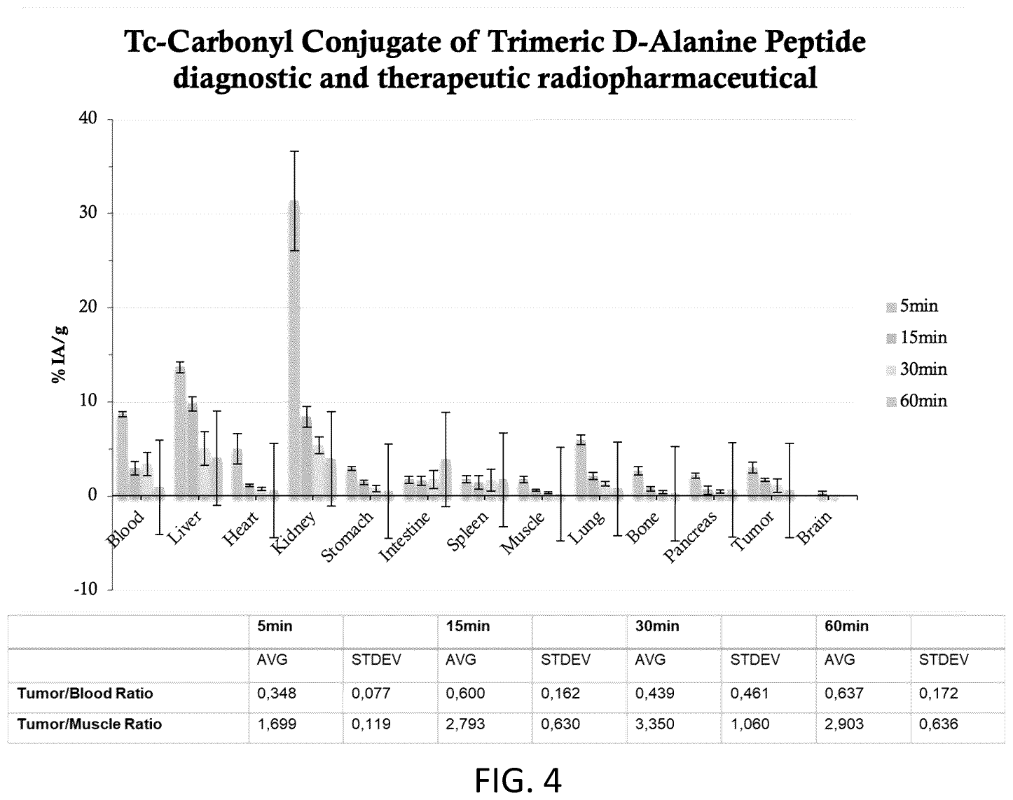

FIG. 4 is data Accumulation of Tc-Carbonyl radiolabeled Trimeric D-Alanine peptide diagnostic and therapeutic radiopharmaceutical in various organs and tissues of breast tumor bearing mice at each time point (Expressed as the mean percentage of Injected Dose per gram±standard deviation (% ID/g±SD)):

Table 2: Biodistribution of Tc-Carbonyl radiolabeled Trimeric D-Alanine peptide diagnostic and therapeutic radiopharmaceutical in organs and tissues of breast tumor bearing mice at each time point (Expressed as the mean percentage of Injected Dose per gram±standard deviation (% ID/g±SD))

| | 5 min | | 15 min | | 30 min | | 60 min | |
|---|---|---|---|---|---|---|---|---|
| | AVG | STDEV | AVG | STDEV | A̅V̅G̅ | STDEV | AVG | STDEV |
| Blood | 8.65 | 0.27 | 2.93 | 0.71 | 3.39 | 1.25 | 0.94 | 0.25 |
| Liver | 13.68 | 0.58 | 9.77 | 0.76 | 5.04 | 1.80 | 3.99 | 0.40 |
| Heart | 4.98 | 1.60 | 1.16 | 0.14 | 0.73 | 0.17 | 0.58 | 0.21 |
| Kidney | 31.33 | 5.27 | 8.38 | 1.10 | 5.37 | 0.90 | 3.95 | 1.10 |
| Stomach | 2.93 | 0.21 | 1.46 | 0.24 | 0.78 | 0.33 | 0.49 | 0.03 |
| Intestine | 1.73 | 0.38 | 1.63 | 0.49 | 1.77 | 0.96 | 3.85 | 0.48 |
| Spleen | 1.76 | 0.37 | 1.44 | 0.70 | 1.68 | 1.15 | 1.72 | 0.62 |
| Muscle | 1.77 | 0.35 | 0.62 | 0.12 | 0.32 | 0.11 | 0.20 | 0.03 |
| Lung | 5.95 | 0.51 | 2.11 | 0.38 | 1.30 | 0.24 | 0.76 | 0.10 |
| Bone | 2.65 | 0.44 | 0.77 | 0.24 | 0.42 | 0.17 | 0.24 | 0.04 |
| Pancreas | 2.16 | 0.29 | 0.63 | 0.43 | 0.49 | 0.19 | 0.67 | 0.30 |
| Tumor | 3.00 | 0.57 | 1.69 | 0.18 | 1.12 | 0.71 | 0.57 | 0.06 |
| Brain | | | 0.29 | 0.19 | 0.09 | 0.03 | | |
| Tumor/Blood | 0.348 | 0.077 | 0.600 | 0.162 | 0.439 | 0.461 | 0.637 | 0.172 |
| ratio | | | | | | | | |
| Tumor/Muscle | 1.699 | 0.119 | 2.793 | 0.630 | 3.350 | 1.060 | 2.903 | 0.636 |
| ratio | | | | | | | | |

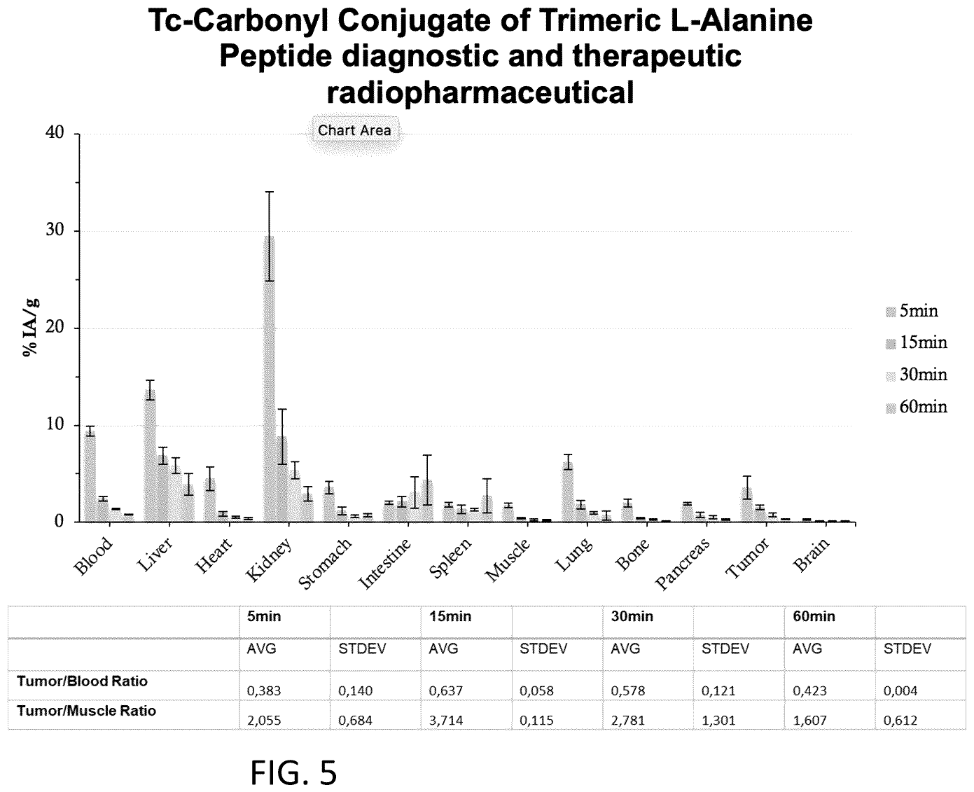

FIG. 5 is data of Accumulation of Tc-Carbonyl radiolabeled Trimeric L-Alanine peptide diagnostic and therapeutic radiopharmaceutical in various organs and tissues of breast tumor bearing mice at each time point (Expressed as the mean percentage of Injected Dose per gram±standard deviation (% ID/g±SD)):

Table 3: Biodistribution of Tc-Carbonyl radiolabeled Trimeric L-Alanine peptide diagnostic and therapeutic radiopharmaceutical in organs and tissues of breast tumor bearing mice at each time point (Expressed as the mean percentage of Injected Dose per gram±standard deviation (% ID/g±SD))

| | 5 min | | 15 min | | 30 min | | 60 min | |
|---|---|---|---|---|---|---|---|---|
| | AVG | STDEV | AVG | STDEV | AVG | STDEV | AVG | STDEV |
| Blood | 9.39 | 0.51 | 2.40 | 0.23 | 1.36 | 0.05 | 0.82 | 0.02 |
| Liver | 13.60 | 1.02 | 6.85 | 0.86 | 5.86 | 0.81 | 3.88 | 1.11 |
| Heart | 4.49 | 1.24 | 0.90 | 0.24 | 0.54 | 0.08 | 0.39 | 0.11 |
| Kidney | 29.45 | 4.59 | 8.81 | 2.82 | 5.38 | 0.89 | 2.91 | 0.73 |
| Stomach | 3.60 | 0.64 | 1.18 | 0.41 | 0.62 | 0.13 | 0.74 | 0.15 |
| Intestine | 2.01 | 0.18 | 2.13 | 0.54 | 3.05 | 1.61 | 4.36 | 2.59 |
| Spleen | 1.81 | 0.23 | 1.37 | 0.44 | 1.31 | 0.14 | 2.72 | 1.73 |
| Muscle | 1.75 | 0.22 | 0.41 | 0.05 | 0.28 | 0.11 | 0.23 | 0.09 |
| Lung | 6.21 | 0.75 | 1.83 | 0.44 | 0.98 | 0.12 | 0.67 | 0.47 |
| Bone | 2.00 | 0.42 | 0.43 | 0.09 | 0.30 | 0.08 | 0.10 | 0.02 |
| Pancreas | 1.93 | 0.13 | 0.73 | 0.27 | 0.55 | 0.18 | 0.26 | 0.07 |
| Tumor | 3.57 | 1.19 | 1.53 | 0.23 | 0.79 | 0.20 | 0.35 | 0.00 |
| Brain | 0.29 | 0.06 | 0.15 | 0.04 | 0.12 | 0.03 | 0.11 | 0.01 |
| T/B | 0.383 | 0.140 | 0.637 | 0.058 | 0.578 | 0.121 | 0.423 | 0.004 |
| T/M | 2.055 | 0.684 | 3.714 | 0.115 | 2.781 | 1.301 | 1.607 | 0.612 |

### SPECT Imaging Studies

Mice were intravenously injected with Tc-Carbonyl radiolabeled Trimeric D-Alanine. SPECT imaging was performed with Molecubes software. A double bed set-up for imaging two mice simultaneously was used. In SPECT software first the field of view (FOV) was adjusted with the desired area of imaging (e.g. whole body). SPIRAL protocol was used for the acquisition of each mouse. The isotope used was selected (e.g. Tc^{99m}) and the activity of the mouse from the dose calibrator was filled in MBq. The specific time of injection is also added. The total acquisition time for the static study was defined (e.g. 45 min). For the image reconstruction voxel size of 500 μm resolution and 500 iterations for better image quality were selected.

CT image acquisition with Molecubes X-CUBE software was performed. Double bed for imaging two mice simultaneously was used. In CT software the field of view (FOV) was adjusted with the desired area of imaging (e.g. whole body). The protocol used is the General-purpose (GP) protocol which is using a medium dose, duration and resolution with a detection level of contrast concerning soft tissues. CT reconstruction conducted using the Iterative algorithm that is regularized statistical image reconstruction using non-negative least squares with higher reconstruction time and better resolution. The voxel size was set at 200 μm.

The quantification process for the SPECT/CT 3D acquisition is conducted with VivoQuant software. The desired data were loaded in the software, first the CT and after the SPECT dem file. For the correct alignment of the two images the Reorientation/Registration tool was used if needed. The Min/Max tool was used to adjust the contrast of the images for better observation of the tumor results. This is important because of the low activity that was observed in the tumor. The image was filtered by using the Gauss algorithm with a value of 2.5 for reducing background and better image presentation. ROI tool was used to draw the desired area for quantification in 3D mode. The connected thresholding algorithm was used to draw the whole mouse and get the counts of the mouse body which is the sum value from the NM in the exported table. The desired areas for quantification (e.g. tumor, liver, lungs) were drawn by hand. The sum value of each drawn area was recorded.

For the correct calculation of the animal injected activity at the specific time point (e.g. 1 h) we are using the well-known formula of or decay correction using the following parameters.
Isotope t_{1/2} (hrs)
Time point (hrs)

Total mouse counts in image (calculated above) for the specific time point.
Dose Calibrator mouse activity (uCi) for the specific time point
Injected activity (uCi) for the specific time point
Sum counts/Organ (calculated above with the ROI)

At last, % ID/Organ or % ID/gram (decay corrected) at the specific time point was calculated by the ratio of each organ to whole-body uptake.

| SPECT post processing | |
|---|---|
| (Vivoquant) | |
| Filter | Gaussian 2.5 |
| Scale bar | To be adjusted |
| | so that signal |
| | from the tumor |
| | can be visible |

For image representation Whole body Maximum intensity projection (MIP) images were extracted with VivoQuant software.

For better observation of selected areas as the tumor in each mouse a 2D, 2 plane image was extracted with 2 planes of selected areas area for every mouse (coronal and transversal).

The following figures illustrate tumor specific targeting ability of both D and L-Trimeric Alanine Peptide labeled Tc carbonyls-thus validation that this new generation of SPECT imaging agents can be used to image and diagnose breast and related neoplastic diseases.

### Results

We present here the chemical architectures of D and L Alanine Peptides diagnostic and therapeutic radiopharmaceutical, their radiolabeling protocols with ^{99m}Tc and details of preclinical investigations using tumor-bearing SCID mice bearing 4T1 breast cancer xenografts, and comparisons of the in vivo kinetics of the radiolabeled amino acids are discussed. The SPECT images of breast tumors from Tc-carbonyl conjugates of both D- and L-Trimeric Alanine Peptide diagnostic and therapeutic radiopharmaceutical provide compelling evidence of tumor specificity of this new generation of SPECT imaging agents. These results, for the first time, demonstrate that alanine receptors on breast and various tumors can be targeted using D- and L-Trimeric Alanine Peptide diagnostic and therapeutic radiopharmaceutical provides tumor cell metabolisable peptides that can serve as SPECT imaging agents. The experiments shows efficient metabolism of both D- and L-Trimeric Alanine Peptide diagnostic and therapeutic radiopharmaceutical by tumor cells. The in vivo SPECT imaging of breast tumors in breast tumor bearing.

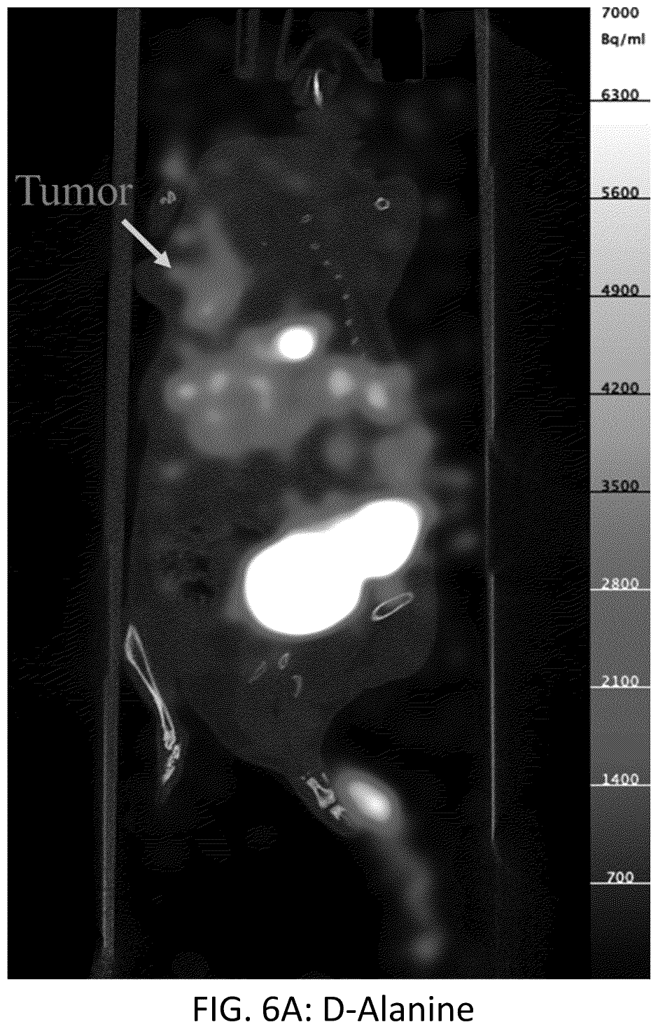

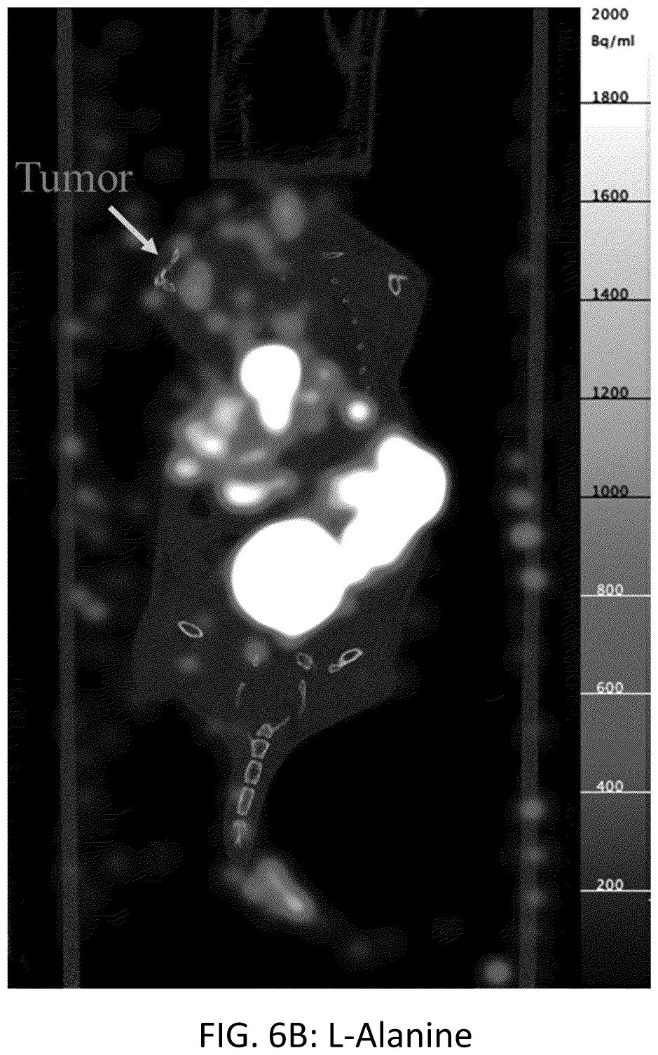

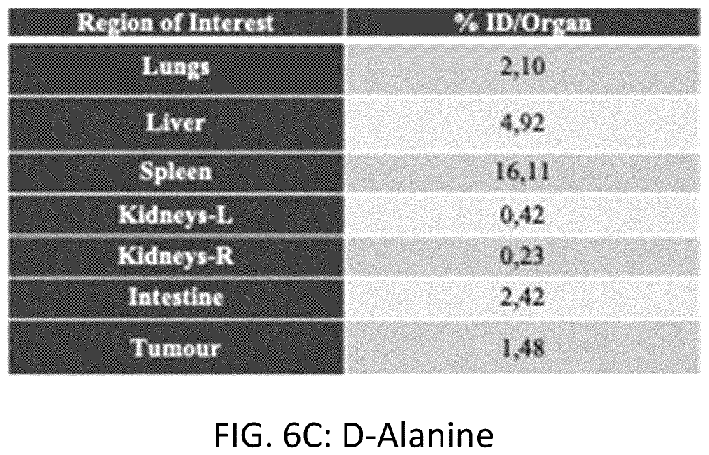

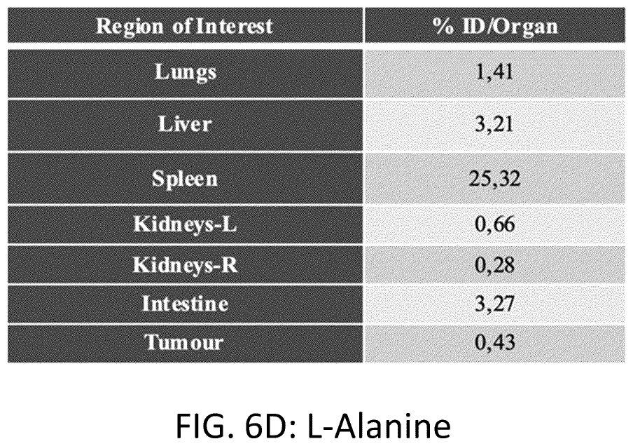

FIGS. 6A-6B are respective SPECT images of breast tumors showing clear accumulation of (FIG. 6A) Tc-Carbonyl conjugate of Trimeric D-Alanine Peptide diagnostic and therapeutic radiopharmaceutical and (FIG. 6B) Tc-Carbonyl conjugate of Trimeric L-Alanine Peptide diagnostic and therapeutic radiopharmaceutical. FIGS. 6C-D show tables of the percentage of the injected dose of the radiolabeled compounds which accumulates in each organ, after intravenous injection of the radiolabeled compound via the tail vein of each mouse, as estimated after the in vivo imaging experiments. The Tables of FIGS. 6C and 6D represents the percentage of the injected dose of the respective D-Alanine and L-Alanine diagnostic and therapeutic radiopharmaceutical which accumulates in each organ, after intravenous injection via the tail vein of each mouse (% ID/organ stands for percent Injected Dose per organ. The numbers on the right are the % ID/organ for as estimated after the imaging experiment. The numbers were estimated from an ex vivo biodistribution study (FIGS. 4 and 5), after each organ was excised and weighed.

The Technetium-(and other imaging isotope) labeled Trimeric D and L-Alanine Peptide diagnostic and therapeutic radiopharmaceutical and similar SPECT probes which can be developed with numerous other Trimeric peptides as will serve as tumor specific SPECT imaging agent.

Although the L-isomer of alanine is incorporated into biological proteins representing second only in abundance to leucine, the higher tumor: blood and tumor: muscle ratios at 60 min p.i. observed for [^{99m}Tc] Tc-D-trimeric Alanine peptide (FIG. 4), in comparison to [^{99m}Tc] Tc-L-trimeric Alanine peptide (FIG. 5), is noteworthy. The experiments provide evidence of utility of chemical architectures of Trimeric D and L Alanine Peptide diagnostic and therapeutic radiopharmaceutical having a SPECT ^{99m}Tc probe. Specifically, D- and L-Trimeric Alanine Peptides, as tumor cell metabolisable peptide diagnostic and therapeutic radiopharmaceutical showed accumulation in targeted organs and cancers to have utility as SPECT imaging agents for diagnosis of various human cancers. The efficient metabolism of both D- and L-Trimeric Alanine Peptide diagnostic and therapeutic radiopharmaceutical by tumor cells can be used as an effective targeting strategy to produce breast tumor (and various human tumors) as specific SPECT imaging agents.

Radiotherapeutic Applications of Peptides Radiolabeled with Actinium-225.

Separate experiments used utilized alpha emitting 225Ac as a radio label for a diagnostic and therapeutic radiopharmaceutical of the invention. The radio label has a half-life of 9.9 days. It decays via a cascade of six short-lived radionuclide daughters to near stable ²⁰⁹Bi (T_{1/2}=1.9×10¹⁹ y). Decay path of 225 Ac yields net four alpha particles with energies ranging from 5.8 to 8.4 MeV and associated tissue ranges of 47 to 85 μm. Additionally, the decay cascade of this alpha emitter includes two beta disintegrations of 1.6 and 0.6 MeV maximum energy. Gamma co-emissions useful for in vivo imaging are also generated in the ²²⁵Ac decay path from the disintegration of ²²¹Fr (218 keV, 11.6% emission probability) and ²¹³Bi (440 keV, 26.1% emission probability).

Highly multidentate Trimeric L-Arginine Peptide was labelled the alpha-emitting isotope Actinium-225 (²²⁵Ac) to form a diagnostic and therapeutic radiopharmaceutical of the invention.

In a low-protein binding Eppendorf, we added 500 μL sodium acetate buffer (labeling buffer, pH 5.6), 55 μL peptide (concentration: 1 mg/mL) and 30 μL ²²⁵Ac (from solid ²²⁵Ac-nitrate dissolved in the labeling buffer). The added ²²⁵Ac activity was approximately 90 kBq for each sample. The mixture was heated at 70° C. for 2 h.

Evaluation of radiolabeling yields: The percentage of radiolabeling yield was determined by ITLC-SG using sodium citrate 0.4M as the mobile phase. 10 μL of the labeled complex were placed at the application point of an ITLC-SG strip (1.5×10 cm), which was then allowed to dry. The free (uncomplexed) ²²⁵Ac migrates to the front (R_{f}=0.8−1.0) of the ITLC strip leaving the labeled complex ²²⁵Ac-peptide at the origin (R_{f}=0.0−0.2). Radiolabeling yield for L-Arginine was ˜32%. Increased yields can be optimized with variations of radiolabeling conditions.

This shows that the Actinium-225 radio label can also be used to form highly multidentate trimeric L-Arginine Peptide diagnostic and therapeutic radiopharmaceutical. Which affords kinetic and thermodynamic stability to Actinium-225 complexes to provide a useful alpha emitting radiotherapeutic agents.

### REFERENCES

[1] M. Nachef, A. K. Ali, S. M. Almutairi and S. H. Lee; Targeting SLC1A5 and SLC3A2/SLC7A5 as a Potential Strategy to Strengthen Anti-Tumor Immunity in the Tumor Microenvironment; Front. Immunol. Sec. Molecular Innate Immunity; Volume 12; pp 1-11, 2021;
[2] D. E. Berning, K V. Katti, C L. Barnes and W. A. Volkert; Chemical and Biomedical Motifs of the Reactions of Hydroxymethylphosphines with Amines, Amino Acids, and Model Peptides J. Am. Chem. Soc. 1999, 121, 1658-1664.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A diagnostic and therapeutic radiopharmaceutical comprising or consisting of a trimeric amino acid structured peptide with a radiolabel.

2. The diagnostic and therapeutic radiopharmaceutical of claim 1, consisting of the trimeric amino acid structured peptide with a radiolabel.

3. The diagnostic and therapeutic radiopharmaceutical of claim 2, wherein the radiolabel is [99mTc] [Tc(H2O)3(CO)3]⁺.

4. The diagnostic and therapeutic radiopharmaceutical of claim 1, wherein the radiolabel is [99mTc] [Tc(H2O)3(CO)3]⁺.

5. The diagnostic and therapeutic radiopharmaceutical of claim 4, wherein the trimeric amino acid is consistent with: 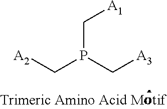
where A₁, A₂ and A₃ are amino acids and P is phosphorous.

6. The diagnostic and therapeutic radiopharmaceutical of claim 5,
wherein A₁=A₂=A₃.

7. The diagnostic and therapeutic radiopharmaceutical of claim 6, wherein the trimeric amino acid is one of a Glycine, Alanine and Arginine amino acids.

8. The diagnostic and therapeutic radiopharmaceutical of claim 2, wherein the radiolabel is alpha emitting 225Ac.

9. The diagnostic and therapeutic radiopharmaceutical of claim 1, wherein the radiolabel is alpha emitting 225Ac.

10. The diagnostic and therapeutic radiopharmaceutical of claim 9, wherein the trimeric amino acid is consistent with: 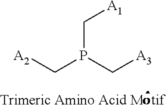
where A₁, A₂ and A₃ are amino acids and P is phosphorous.

11. The diagnostic and therapeutic radiopharmaceutical of claim 10, wherein A₁=A₂=A₃.

12. The diagnostic and therapeutic radiopharmaceutical of claim 11, wherein the trimeric amino acid is one of a Glycine, Alanine and Arginine amino acids.

13. A method for SPECT imaging, comprising:
introducing diagnostic and therapeutic radiopharmaceutical in accordance with claim 1 into an animal;
imaging one or more organs in the animal.
